(19) United States
(12) Patent Application Publication
(10) Pub. No.: US 2016/0331396 A1
(43) Pub. Date: Nov. 17, 2016

(54) SURGICAL HANDLE FOR A TOOL HAVING A TUBULAR SHIFT

(71) Applicant: AESCULAP AG, Tuttlingen (DE)

(72) Inventors: Tom Schweitzer, Tuttlingen (DE); Wolfgang Sauter, Renquishausen (DE)

(21) Appl. No.: 15/112,880

(22) PCT Filed: Jan. 14, 2015
(86) PCT No.: PCT/EP2015/050538

§ 371 Date: Jul. 20, 2016

(30) Foreign Application Priority Data
Jan. 21, 2014 (DE) 10 2014 100 603.8

(51) Int. Cl. A61B 17/32

(52) U.S. Cl. CPC A61B 17/320016; A61B 2017/320064; A61B 2017/00862

(57) ABSTRACT

A surgical handle for a surgical instrument, in particular a shaft-type instrument for actuating a tool that is distally arranged on an instrument shaft, includes a handle housing on which at least one handle is mounted so as to be movable relative to the handle housing. The handle is coupled via a toggle mechanism to a sliding element that is axially movable in the handle housing in order for the sliding element to be moved as the handle is actuated from the starting position against the restoring force of a spring element, the spring element being embodied by the toggle mechanism.

### RELATED APPLICATIONS

This application is the United States National Phase entry of International Application No. PCT/EP2015/050538, filed Jan. 14, 2015, which is related to and claims the benefit of priority of German Application No. DE 10 2014 100 603.8, filed Jan. 21, 2014. The contents of International Application No. PCT/EP2015/050538 and German Application No. DE 10 2014 100 603.8 are incorporated by reference herein in their entireties and for all purposes.

### FIELD

The present invention relates to the field of surgical instruments, especially to an axial handle for surgical instruments having a tubular shaft and to a surgical instrument preferably of the minimally invasive type.

### BACKGROUND

In the field of surgery operations are frequently carried out in a way referred to as “minimally invasive”. Operations inside the body are in this case carried out by small incisions and by means of appropriate usually sophisticated instruments which are operated externally, i.e. from outside of the patient's body by the operating surgeon. For the patient this means less strain during operation, as no big cuts have to be carried out, i.e. the wounds are kept very small. This method is applied in cardiosurgery, laparoscopy (intra-abdominal examination), arthroscopy, for example. Manipulative operations such as the collection of tissue specimens or the removal of organs (e.g. removal of the gall bladder) are possible by this method.

For this purpose, so called instruments having a tubular shaft are utilized. Such instrument having a tubular shaft usually includes a preferably hollow instrument shaft to the distal end of which a (tubular shaft-type) tool, for example in the form of scissors, fixation forceps, needle holder or similar mechanically operable actuators, is articulated by which the operating surgeon may operate on the patient. For this, at the proximal end of the instrument shaft a device in the form of an operating handle is provided which enables a force/moment that has to be applied for actuating the tool to be introduced to a force transmission train preferably inside the instrument shaft, the force transmission train then transmitting the introduced force/moment to the tool to actuate the same.

There exist specific operating handles in the form of handles which are adapted to be coupled to the described instruments or are fixedly mounted thereto for actuating the tool. High requirements in terms of cleaning, dismounting, stability or tactile behavior are made to such handles.

A surgical handle for a surgical instrument of the afore-mentioned design usually consists of a handle housing which is connectable/connected to the instrument shaft and accommodates a mechanism for force/moment transmission to the force transmission train operatively connected to the tubular shaft-type tool, of levers/handles articulated to the handle housing which are manually operable and are in operative connection with the mechanism, of a distal connecting device, where appropriate, via which the handle can be connected to the instrument shaft and the force transmission train can be connected to the mechanism inside the housing and of a gear system preferably in the form of toggle means which couples the levers/handles to the mechanism inside the case for actuating the latter.

In case that the handle is a pivoted lever articulated to be pivoting on the handle housing, a toggle is articulated at one end thereof to a central portion of the pivoted lever, wherein the other end thereof is articulated to a sliding element or push/pull rod supported to be axially movable in the handle housing which sliding element in turn is coupled to the force transmission train (e.g. a push/pull rod or Bowden cable inside the instrument shaft).

When the pivoted lever is manually pivoted toward the instrument housing, an axial displacement is imparted to the sliding element via the toggle to actuate the distal instrument tool. For returning the pivoted lever into its starting position, on the sliding element and/or on the pivoted lever itself a return spring is arranged which is tensioned upon manual operation of the pivoted lever and returns the pivoted lever when it is released.

A possibility of generating the restoring force for the handles resides in an axial compression spring (coil spring) which is installed in the handle housing and axially resets the sliding element to the initial position when the operating force manually applied to the handle is reduced. The axial compression spring installed in the handle housing requires space, however. Apart from that, the spring force acts only downstream (distally) of the toggle transmission, viewed in the direction of force. In this way, depending on the angular position of the toggle and thus of the operating handle, the impact of the spring force is strongly reduced. When the tool is almost closed, the impact of the spring force is strongly dropped and may result in the tool snapping shut in an undesired as not controllable manner.

Therefore, handles available on the market having the afore-described conceptual design exhibit various restrictions. For example, the handles for tubular shaft-type instruments frequently cannot be dismounted due to their complicated mechanism. Apart from that, the tactile behavior with available handles having the afore-described structure is not optimal or gets even largely lost, because the spring is connected downstream of the toggle mechanism.

A second variant consists in the arrangement of a spring on the handle itself, i.e. between the handle housing and the operating handle, as it is also realized in known scissors designs, for example. The spring force in this case acts directly between the handle housing and the operating handle and thus upstream of the toggle mechanism when viewed in the direction of force. In this case the handle must have high rigidity, however, which accordingly entails high weight.

WO 2013/079340 discloses a surgical handle in which a sliding element is axially displaced inside a handle housing by means of a joint element in the form of a toggle so as to actuate a downstream force transmission train. Handles/levers that are engaged in the joint elements are forced toward each other and act on the sliding element via the joint elements. The restoring force acting on the handles is generated by an axial compression spring that is seated in the handle housing and acts on the sliding element. In addition, the handle includes stop elements axially acting on the sliding element for restricting the actuating path thereof that are intended to prevent the toggle mechanism from reversing. This means a mounting effort for the additional parts, higher weight and more gaps that impede cleaning of the device.

US 2008/064929 A1 discloses an axial handle for a tubular shaft-type instrument in which the restoring force acting on the actuating levers is generated via leaf springs each of which forms an integral part or a longitudinal portion of the actuating levers. The actuating levers are solid and consequently heavy which is a drawback in handling the instrument during an operation.

### SUMMARY

In the following sections, the term “proximal” is used as “being close to the handling person” and the term “distal” is used as “being distant from the handling person”.

It is the object of the invention to avoid the afore-mentioned drawbacks and to provide a surgical handle as well as a surgical instrument which provides clear tactile feedback to the operator, offers the option of simple dismounting and can be easily cleaned.

This object is achieved by a surgical handle comprising features described herein as well as by a surgical instrument comprising the features described herein.

According to a first aspect, the core of the present invention consists in the provision of a surgical handle for a surgical instrument, especially a shaft-type instrument, for actuating a tool arranged distally on an instrument shaft comprising a handle housing to which at least one lever/handle is mounted to be movable/pivoting relative thereto, the lever/handle being coupled via a toggle mechanism to a sliding element that is axially movable in the handle housing so as to move the sliding element by operating the lever/handle from the starting position thereof against the restoring force of a spring element and in this way to operate the tool. In accordance with the invention, the toggle mechanism is constituted by a resilient element, or in other words, the toggle lever simultaneously forms the resilient element for generating a restoring force on the lever/handle.

Due to this measure, the arrangement of an additional separate spring element for restoring the lever/handle may be dispensed with, which saves space in the design that can be used for other purposes, ensures simpler dismounting of the handle and guarantees better cleaning of the handle and thus of the instrument by a reduced number of narrow interstices.

It is favorable when the surgical handle comprises a first lever/handle which is swivel-mounted about a pivot axis aligned transversely to the handle axis on the handle housing. The swivel-mounting of the first lever/handle permits the clearly defined and user-friendly movement thereof relative to the handle housing. In order to achieve a compact design of the handle the first lever/handle is swivel-mounted directly on the handle housing. The term “transversely to the handle axis” means that it is provided in a plane perpendicularly to which the handle axis is oriented.

Concerning the handling it is advantageous when the first lever/handle is swivel-mounted with its proximal end and is pivoting relative to the handle housing with its distal free end. In this way the first lever/handle may be seized and operated more easily by the user.

Preferably the first lever/handle is swivel-mounted on or close to a proximal end of the surgical handle, for in this way it is possible to impart a compact design to the surgical handle.

The first lever/handle preferably can be transferred from a braced position relative to the handle housing which it adopts in the non-operated position (starting position) to an approximated position relative to the handle housing which it adopts in the at least one operating position, and vice versa. For operating the surgical handle the first lever/handle may be transferred from the braced position to the approximated position while pivoting relative to the handle housing. This facilitates a user's handling of the surgical handle.

It is favorable when the first lever/handle can be fixed in at least one actuating position, for this entails the possibility of fixing also the sliding element and thus the force transmission element at one position. For example, this is favorable when claw parts for seizing body tissue or a surgical instrument such as a needle are arranged at the distal end of the tubular shaft-type tool.

The force is generally transmitted to the tubular shaft-type tool via the toggle transmission. In a conventional toggle transmission the transmitting element between the handle and the sliding element is configured to be non-resilient, therefore the resetting of the lever/handle has to be generated via an additional elastic element, preferably a spring. Hence it is intended to manage on fewer components and to avoid the drawbacks of a rigid toggle lever.

The invention so-to-speak combines the function of the toggle lever that is rigid per se with the characteristic of the resilient element. In other words, this element unifies the characteristics of the rigid toggle and the spring within one component, thus providing additional space in the handle housing which can be used otherwise, for example for improving the coupling device to the tubular shaft-type instrument. This saves manufacturing costs during assembly and facilitates cleaning due to fewer gaps.

The combination of the two functions of a toggle transmission and a return spring is taken over, according to the invention, by one single component, preferably a leaf spring that replaces the rigid toggle lever according to prior art.

A leaf spring in general has a substantially elongate shape including first and second ends. At least at one end the leaf spring may include means for fastening by which it can be fastened to a counter piece, preferably the lever/handle and/or the sliding element.

The means for fastening to one or both ends of the leaf spring may be configured so that they ensure a rigid connection relative to the object to which they are fastened or else a movable/pivoting connection relative to the object to which they are fastened.

Said means may be, for example, holes for screws and rivets, thickened portions of the material at ends of the leaf spring, widened portions, ends of the leaf spring bent to form eyelets or similar means.

The object (sliding element/handle) to which the leaf spring may be fastened itself provides means that correspond to said means at the ends of the leaf spring and support a connection to the fastening means at the ends of the leaf spring.

Said means may be for example screw threads, axes of rotation, clamping slits, clamping slits including a blind hole, groove/tongue connection and similar means which are suited for connecting the end of the leaf spring to the object.

Moreover, adhesive, soldering and welding connections may be added, for the purpose of assistance or on their own, to the afore-mentioned means so as to connect the end of the leaf spring to an object (sliding element/handle).

Preferably the leaf spring is expanded linearly and along a longitudinal axis. The width of the leaf spring is larger than the thickness/height.

The thickness of the leaf spring may be selected independently along the length of the leaf spring to achieve a desired spring characteristic.

The width of the leaf spring is homogenous at least in portions.

The leaf spring may have areas in which the width/thickness ratio of the spring varies so as to achieve different spring characteristics.

For assembly it may be favorable when the width of the leaf spring decreases toward one or both ends.

The leaf spring may also take a curved shape in the stress-free state so that an (imaginary) connection between the ends of the leaf spring is shorter than the length of the leaf spring itself (arc shape).

The radius of curvature of the leaf spring may be homogenous in portions. Equally, the curvature of the leaf spring may have plural different radii of curvature (while maintaining the arc shape) so as to obtain protection against overload, for example.

The handles or else levers or else handle shells include upper and lower surfaces, wherein an upper surface is facing the palm of a hand and a lower surface is facing away from the palm. Each lever/handle includes means for introducing a force to the leaf spring so as to deform the leaf spring in order for the latter to store energy required for resetting the handle into an initial position.

These means may be arranged on the surface facing away from the palm, i.e. on the surface of the handles facing the handle housing. The force application means may act on a spot or the full surface of the leaf spring in the central portion thereof, for example such that an initially pre-curved (arc-shaped) leaf spring is resiliently deformed with increasing actuation of the lever/handle into the straight position so as to return to the curved shape again when the lever/handle is released and accordingly to swivel the lever/handle back into the initial position (starting position) and at the same time to withdraw the sliding element. The force application means may adopt the shape of a projection, for example, on the respective lever/handle, the projection being adjacent to a central portion of the leaf spring (resilient toggle lever) and pushing the latter through upon actuation of the lever/handle, or may be sort of a link guide along which the leaf spring rolls off and accordingly expands.

The force application means may contact the leaf spring without any force application when the handles are unloaded, i.e. in the initial state.

The leaf spring according to the invention may be formed of suitable elastic materials such as e.g. spring steel, plastic material, fiber-reinforced plastic material.

The fact that upon compressing the handle a continuously increasing spring tension is generated renders an additional compression spring superfluous. By an appropriate shape of the leaf spring an overload protection can be additionally achieved which is especially desired for protecting the sophisticated instruments.

For example, the leaf spring may be curved more strongly compared to the arc shape at a spring end portion preferably in the area of the sliding element (radius is narrower than in the residual spring portion) so that said spring portion having a smaller bending radius bulges upon reaching or exceeding a particular axial force acting on the leaf spring (according to the general toggle function) and thus restricts the axial force transmission from the lever/handle to the sliding element.

The handle according to the invention preferably comprises a second lever/handle so as to facilitate the user's handling of the handle. Preferably it may be provided that the second lever/handle is immobilized on the handle housing as in this way a simpler constructional design can be imparted to the handle.

The second lever/handle may as well be movable and may be swivel-mounted on the handle housing, analogously to the first lever/handle, about a pivot axis aligned transversely to the handle axis. Furthermore the second lever/handle may be operatively connected to the sliding element by a toggle lever so as to transmit an actuating force (axial force) to the sliding element. Of preference, for connecting the second lever/handle and the sliding element equally the resilient element according to the invention, and in particular a leaf spring, is employed.

When realizing the handle in practice it turns out to be favorable when the first lever/handle and/or the second lever/handle take a shell shape and enclose at least portions of the handle housing especially in sleeve shape in the circumferential direction of the handle axis. The levers/handles are arranged on two opposite sides of the handle axis, for example, and may be seized and operated more easily by the user's palm.

Furthermore, it turns out to be advantageous when each of the first lever/handle and the second lever/handle are configured as axially extending semi-cylindrical or substantially semi-cylindrical shells receiving the handle housing there between.

### BRIEF DESCRIPTION OF THE DRAWING FIGURES

The invention shall be illustrated in detail hereinafter by way of preferred embodiments with reference to the accompanying drawings.

FIG. 1 exemplifies the interaction of the individual parts and the resetting of the lever/handle with the toggle having an axial compression spring,

FIG. 2 illustrates the interaction of the individual parts and the resetting of the lever/handle with the toggle having a return spring between the handle housing and the lever/handle,

FIG. 3 exemplifies an embodiment of a handle according to the invention comprising a resilient toggle,

FIG. 4 exemplifies another embodiment of a handle according to the invention comprising a resilient toggle,

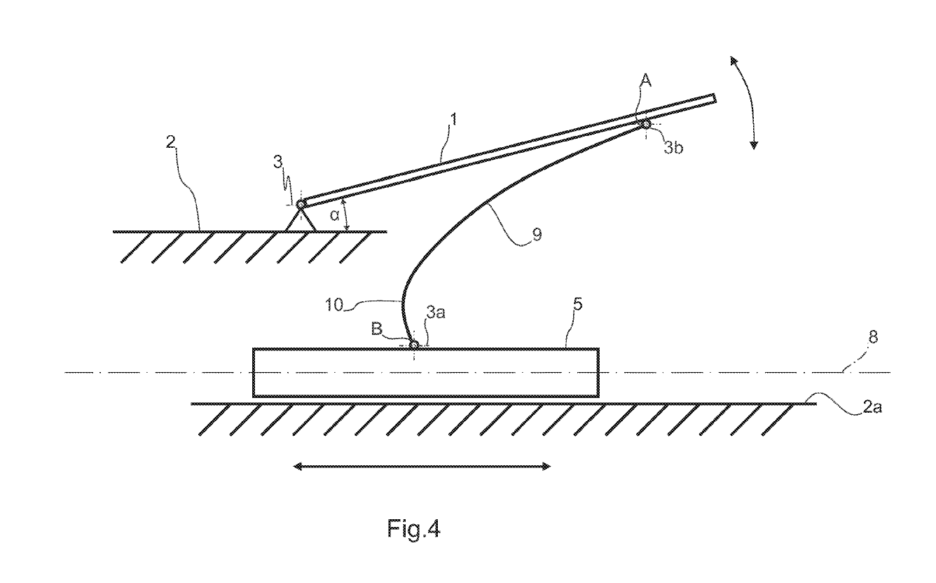

FIGS. 5a-5c illustrate the function of the resilient toggle by way of the embodiment including a curved spring,

FIGS. 6a and 6b illustrate the function of the resilient toggle by way of the embodiment including a non-curved spring,

FIGS. 7a-7d illustrate the various options of fastening of the leaf spring on the sliding element,

FIGS. 8a and 8b show the effect of the overload function,

FIGS. 9a and 9b show in detail the leaf spring according to the invention and the leaf spring having an overload function,

FIGS. 10a-10c illustrate the real surgical handle including the leaf spring according to the invention in the mounted/assembly state in various views, and

FIGS. 11a-11c illustrate the real surgical handle including the leaf spring according to the invention with overload protection in the mounted/assembly state in various views.

### DETAILED DESCRIPTION

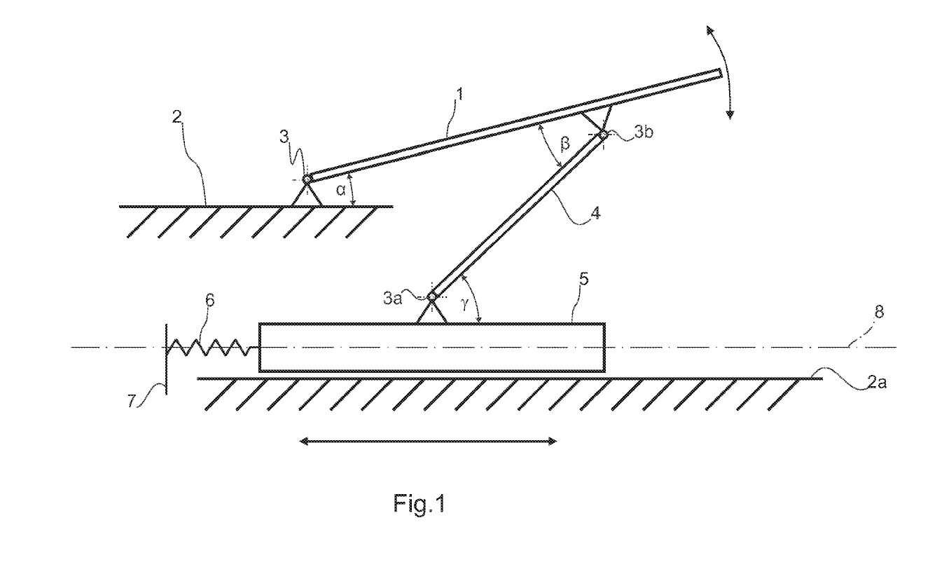

FIG. 1 exemplifies the effect of the resilient toggle in the version known from the state of the art. The operating handle used in the real handle is shown here by a simple lever/operating handle 1. The lever 1 is swivel-mounted to a handle housing, exemplified by the reference numerals 2, 2a, about an axis 3. Preferably, the axis 3 is arranged on a proximal end of the handle.

On a distal end of the lever 1 a toggle 4 is equally swivel-mounted about an axis 3b. Furthermore, the toggle 4 is pivoting relative to a sliding element 5 about an axis arranged on the sliding element 5.

A spring 6 is mounted in a proximal position between a stop 7 in the proximal part of the handle housing 2, 2a and the sliding element 5 which is provided inside the handle housing 2, 2a.

In the exemplary representation of FIG. 1 the orientation of the levers 1, 4 is such that the angle a enclosed between the handle housing 2, 2a and the operating handle 1 opens in the proximal direction and the angle β enclosed by the operating handle 1 and the toggle 4 opens in the distal direction. Thus it is possible to reverse the orientation and consequently to reverse the direction of the idle position (starting position) and the displacement. In this case the spring 6 and the stop 7 are mounted to be mirror-inverted on the other side of the sliding element 5.

In FIG. 1 the current disadvantageous state of the art is illustrated by way of the variant including an axial compression spring as flexible restoring element. Reference numeral 5 denotes the sliding element that causes the coupling of the force introduced via the operating handle 1 and the toggle 4 to the sliding element 5 onto the tubular shaft-type instrument. The tubular shaft-type instrument itself including the pertinent coupling device thereon is not shown.

When the lever 1 is pressed down, the toggle 4 movably connected thereto and thus also the sliding element 5 movably connected to the toggle 4 moves in the axial direction thereof. The lever 4 is referred to as toggle in this arrangement and represents a rigid non-flexible connection.

The more the lever 1 is swiveled about the axis 3 toward the handle housing 2, 2a, the more the toggle 4 moves the sliding element 5 against the force of the spring 6. The spring 6 can apply a restoring force to the lever (the handle part) 1 only as long as the lever 1 and, respectively, the lever 4 do not fall below a minimum angle with respect to the handle axis (parallelogram of forces). It has always to be ensured that an upwardly directed force acts on the lever 1. If the toggle 4 arrived at a position in parallel to the handle axis 8, no more upwardly directed force would be provided and the toggle 4 would remain in its position. The surgical handle in such moment so-to-speak would snap shut and thus also maintain the tool arranged at the distal end of the tubular shaft in the closing position.

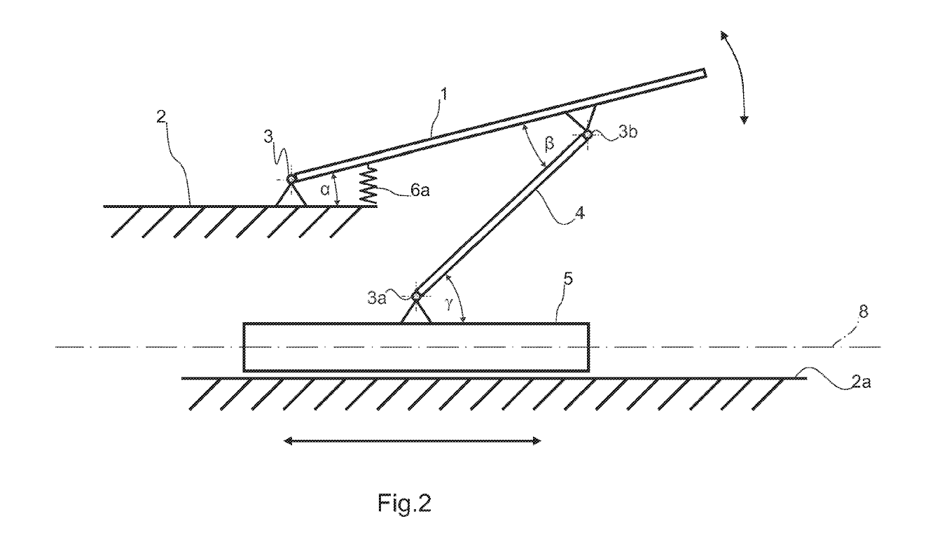

The second equally disadvantageous variant of the state of the art is exemplified in FIG. 2. The return spring 6a is disposed between the operating handle (lever) 1 and the handle housing 2, 2a close to the pivot axis 3. This requires the handle 1 to be rigid, which consequently means that the handle 1 is solid and heavy.

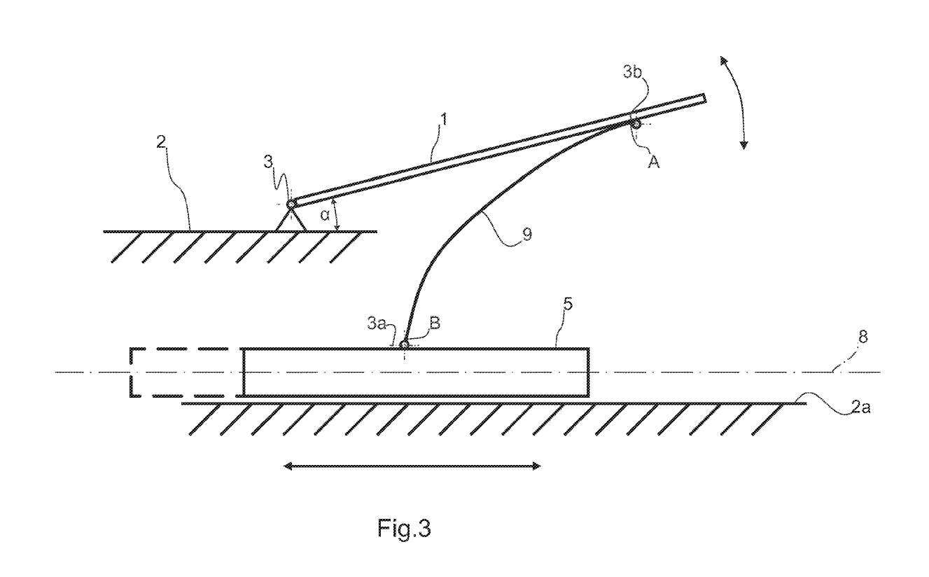

In FIG. 3 the spring element/leaf spring 9 according to the invention is exemplified which basically replaces the rigid toggle as well as the return spring according to the invention arranged separately herefrom. The spring element/leaf spring 9 includes a first end A and a second end B. By the end A it is connected to the lever and, resp., the handle shell 1, by the other end B it is connected to the sliding element 5.

The spring element 9 preferably may be curved (arcuate). When the lever/handle 1 is swiveled about the axis 3 in the direction of the handle housing 2, 2a and the angle enclosed between the lever/handle 1 and the handle housing 2, 2a is reduced, the distance between the ends A and B of the leaf spring 9 is continuously increased. This means to the leaf spring 9 that it is deflected out of the arcuate curved state, which represents its idle position, in the direction of the straight alignment and thus absorbs energy for resetting the handles 1.

The connection both to the sliding element 5 and to the operating handle 1 may be configured as pivot axis (3a, 3b); however, also a rigid connection relative to each of the handle 1 and the sliding element 5 is possible. A combination of both fastening options is imaginable as well.

The expansion of the leaf spring 9 during swiveling the handle lever 1 about the pivot axis 3 in the direction of the handle housing is brought about by the fact that portions of the leaf spring 9 adapt to (roll off) part of the handle 1 facing the handle housing and hence are straightened, i.e. experience an expansion. By the expansion of the leaf spring 9 and by swiveling the lever 1 the sliding element 5 connected to the end of the leaf spring 9 is axially moved along the handle axis 8.

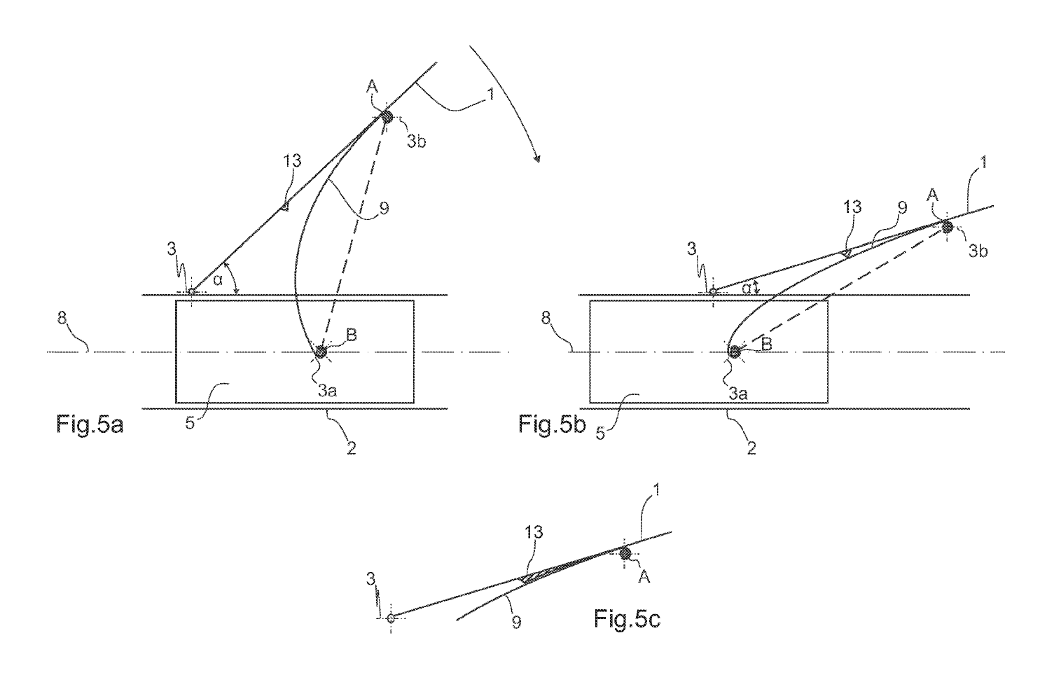

The lower part of the handle 1 facing the handle housing may also include force application means 13 (projection/journal etc.) as shown in FIGS. 5a-5c which may be in the form of projecting structures, for example.

When swiveling the handle 1 about the axis 3, the projecting force application means 13 enters into contact with the leaf spring 9 and upon further swiveling about the axis 3 at this location the force is applied to the leaf spring 9 and causes the afore-described expansion (FIG. 5b). The distance of the force application means 13 from point A and also the structural size thereof determine when and at which pivoting angle of the handle 1 relative to the handle housing the expansion of the spring starts. FIG. 5c shows a handle 1 in which the force application means 13 may be adjacent to portions of the leaf spring 9.

As far as the function of the resilient toggle lever 9 according to FIGS. 5a to 5c is concerned, the following can be stated:

At the start of actuation the shown lever 1 is provided in the swivel-out position and the sliding element 5 is accordingly provided in the retracted position. When based on this fact the lever 1 is swiveled in the direction of the handle housing, the sliding element 5 is displaced over the toggle (C-shaped leaf spring) 9 along the axis 8. The leaf spring 9 continuously rolls at its central portion off the projection 13 which elastically indents and thus straightens the leaf spring 9 in the central portion with an increasing degree of actuation. When the lever 1 is released, the energy stored in the leaf spring 9 causes the leaf spring 9 to automatically return to the pre-curved shape. Accordingly, the lever 1 is swiveled back to its initial position and axially pulls the sliding element 5 back to the original position thereof.

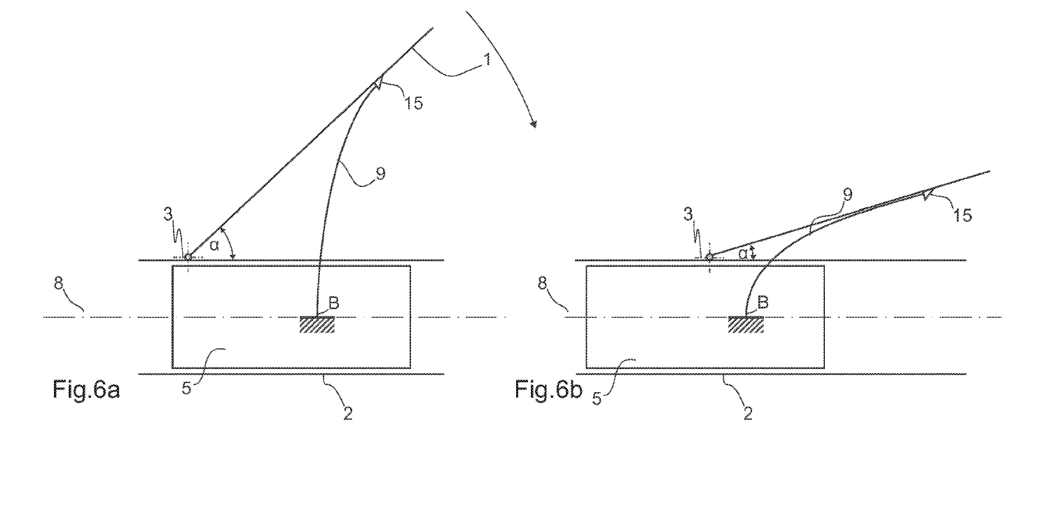

In FIGS. 6a and 6b another variant of the resilient toggle lever is shown. In this example the leaf spring 9 is rigidly coupled to the sliding element 5. Thus the movable bearing 3 between the leaf spring 9 and the sliding element 5 is dropped.

The sliding element 5 and the leaf spring 9 in this case may be a non-dismountable part that is to be delivered in complete form.

In this constellation the leaf spring 9 may preferably be straight. In order to achieve the restoring effect, the terminal points A and B of the leaf spring 9 do not move apart from each other during swiveling of the handle 1 about the axis 3 as in the preceding embodiment, but move toward each other due to increasing elastic bending of the leaf spring 9. The one end A of the leaf spring 9 may abut against a stop 15 arranged on the surface of the handle 1 facing away from the palm or the leaf spring 9 is hinged to the lever 1. The stop 15 (or hinge) prevents the end A of the leaf spring 9 from moving relative to the handle 1 when the handle 1 is compressed. The stop 15 (or hinge) thus ensures the force application and hence the deformation of the leaf spring 9 when the handle 1 is swiveled about the axis 3. At the same time, an axial force is transmitted to the leaf spring 9, thus causing the sliding element 5 to be displaced along the axis 8.

FIG. 6b shows the leaf spring 9 when the handle 1 is compressed. The leaf spring 9 now is curved and, due to the fact that it has stored deformation energy, is capable of returning the handle 1 into the initial position and at the same time to withdraw the sliding element 5 into the original position thereof.

Additional force application means 13 as mentioned in the previous embodiment are not required, but can be added as required, e.g. in order to achieve a particular characteristic in the force path. In order to achieve a particular characteristic of force application it is also possible to arrange the leaf spring 9 at an angle relative to the sliding element 5, wherein the imaginary pivot axis about which said angle rotates is in parallel to the axis 3. The characteristic of force application depends on the respective fixedly selected angle.

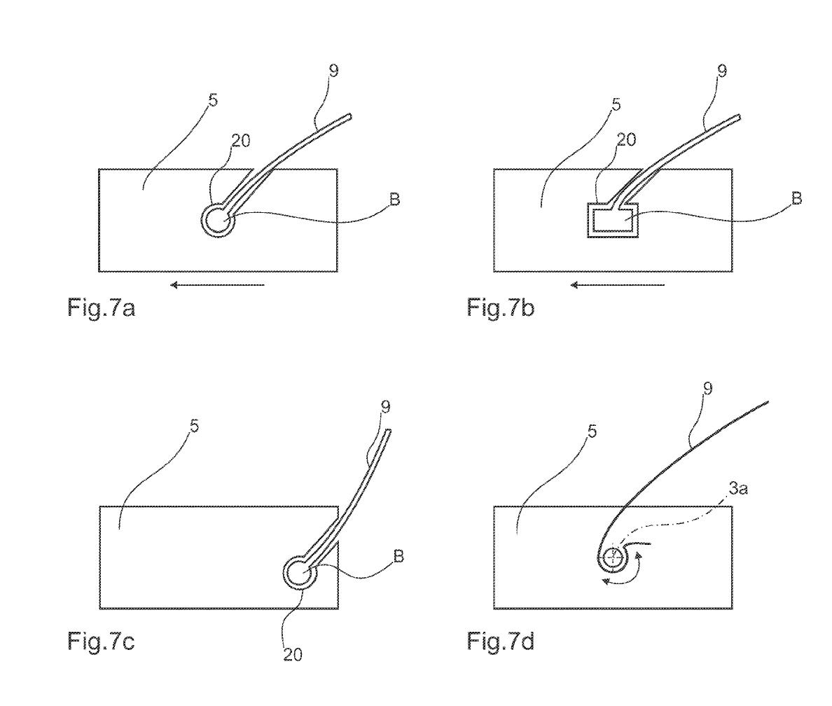

FIGS. 7a-7d illustrate the different possible types of fastening (rigid and non-rigid) of the leaf spring 9 relative to the sliding element 5.

FIGS. 7a-7c show rigid variants, by way of example by connections similar to the groove and tongue system. At the end of the leaf spring 9 various means for connection to the sliding element 5 are provided. The sliding element includes the means corresponding thereto.

Further imaginable is a connection not illustrated here which is only clamped, for example, hence without either of the ends of the leaf spring 9 including any particular fastening means. In the simplest case, the sliding element 5 may have a slit into which the one end of the leaf spring is clamped.

A connection between the leaf spring 9 and the sliding element 5 can be achieved by soldering, gluing or clamping, depending on the material.

FIG. 7d illustrates a movable connection in which one end of the leaf spring 9 is bent to form an eyelet and is movably supported about an axis 3b on the sliding element 5.

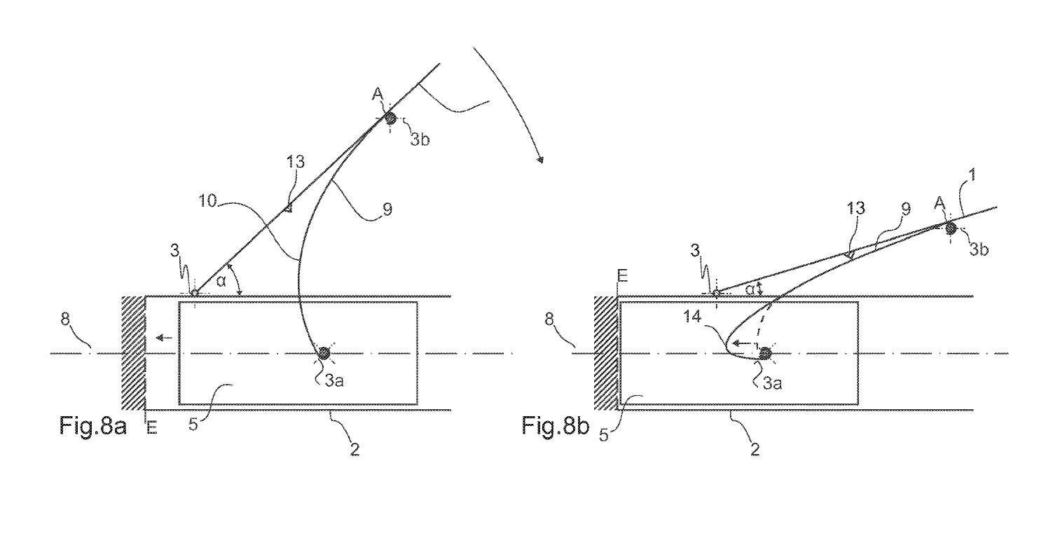

In FIGS. 8a and 8b, another embodiment of the leaf spring 9 according to the invention is shown. As is evident, the leaf spring 9 includes another simple curvature 10 having a radius of curvature that is smaller than that of the residual arc shape. This additional curvature 10 which is clearly visible in FIG. 8a (and, resp., also 9b) has the function of protecting the instrument in the case of overload. Overload may occur when the handles 1 are closed beyond a point at which the tool is closed already on the distal side. This means that, when the branches of forceps, for example, are closed already, the handles 1 can be further compressed and an increasing force is acting on the same. Since the leaf spring 9 continues bending into a straight shape, i.e. continues storing deformation energy the more the handles 1 are compressed, it becomes ever more rigid in the direction of the handle axis 8.

It would reach its maximum rigidity if it were aligned in parallel to the handle axis 8. Although this extreme case cannot occur, as the axis of rotation 3 of the handle 1 is not located on the handle axis 8 but on the handle housing 2, 2a, in this bending state, however, when manual actuating force is further applied to levers/handles 1, the leaf spring 9 can transmit so much force via the gear mechanism to the distal end of the tubular shaft-type tool that the sophisticated tools might be damaged. Damage of the coupling means is also possible at the proximal end of the tubular shaft-type tool.

The additional curvature 10 of the leaf spring 9 in the illustrated area may produce relief. In FIG. 8a the leaf spring 9 is moved in the direction E. In this case E represents a final position at which any further movement of the sliding element 5 in the direction of the handle axis 8 is no longer possible. FIG. 8b shows what happens when the sliding element 5 abuts against the terminal point E.

If the force continues increasing in the direction of the handle axis 8, cf. FIG. 8b, apart from the deformation of the leaf spring 9 building up the restoring force further deformation/bulging 14 of the leaf spring 9 takes place in the area of the additional curvature 10, which is represented here in exaggerated form for the purpose of illustration. The excessive force thus changes its direction and is converted to additional deformation energy. The additional curvature 10 may be referred to as an additional leverage or an additional lever action, respectively, on the leaf spring 9.

When the handle 1 is released or the manual actuating force on the levers/handles 1 is reduced, at first the sliding element 5 does not move back, but the leaf spring 9 initially reduces the additionally obtained energy by reverse-bending the deformation/bulging 14 into the state shown in FIG. 8a, before it returns the sliding element 9 into the initial position and thus also moves the handles 1 into their initial position.

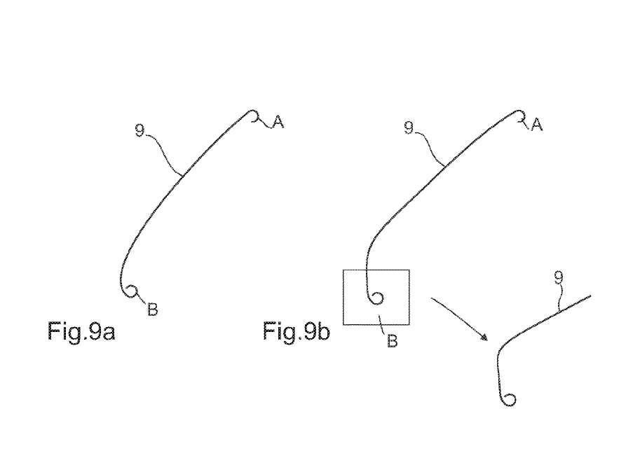

FIGS. 9 and 9a illustrate in detail the leaf spring 9 according to the invention without (FIG. 9) and with overload curvature (FIG. 9a).

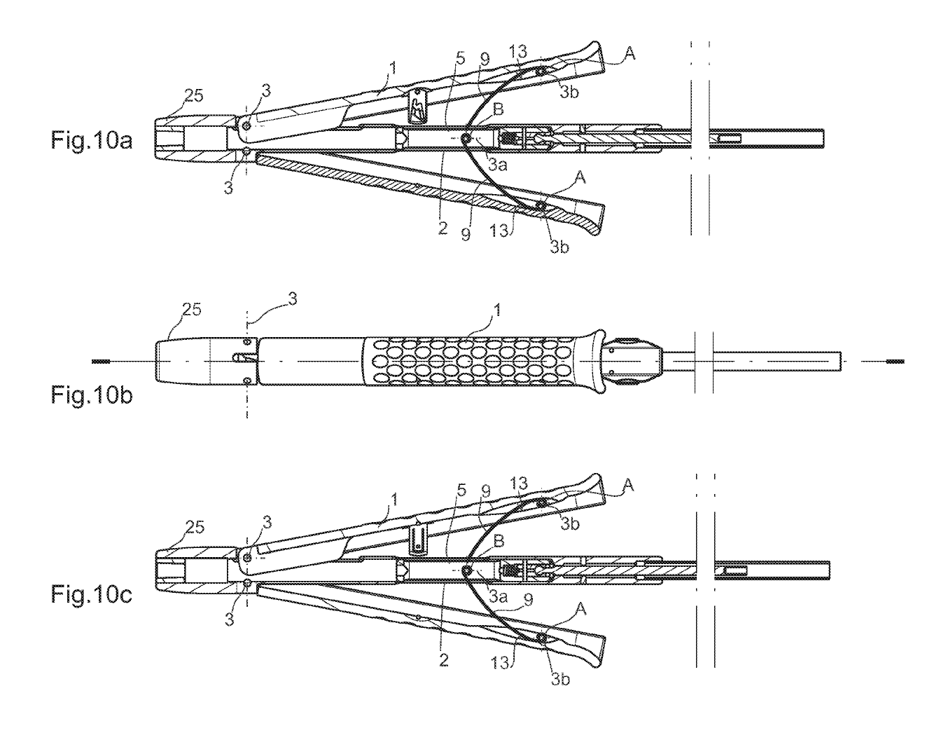

FIGS. 10a-10c show a surgical handle of real design comprising the leaf spring 9 according to the invention in several views. A Luer cone 25 for guiding rinsing liquid there through may be arranged at the proximal end of the surgical handle.

Accordingly, the levers 1 hinged to the handle housing 2 having a cylindrical shape in this case are shaped as lever shells that are extremely rigid. On the sides of the lever shells facing the handle housing 2 link-shaped notches are visible on which the leaf springs 9 pre-bent in C-shape rest in the central portions thereof upon actuation of the levers 1 and in this way are resiliently pressed to become straight. Simultaneously, via the leaf springs 9 a thrust force is transmitted to the sliding element 5 which is supported and guided to be sliding in the handle housing 2. The force transmission mechanism represented in FIG. 10 as a push-/pull rod which is guided in an instrument shaft is coupled to the sliding element 5.

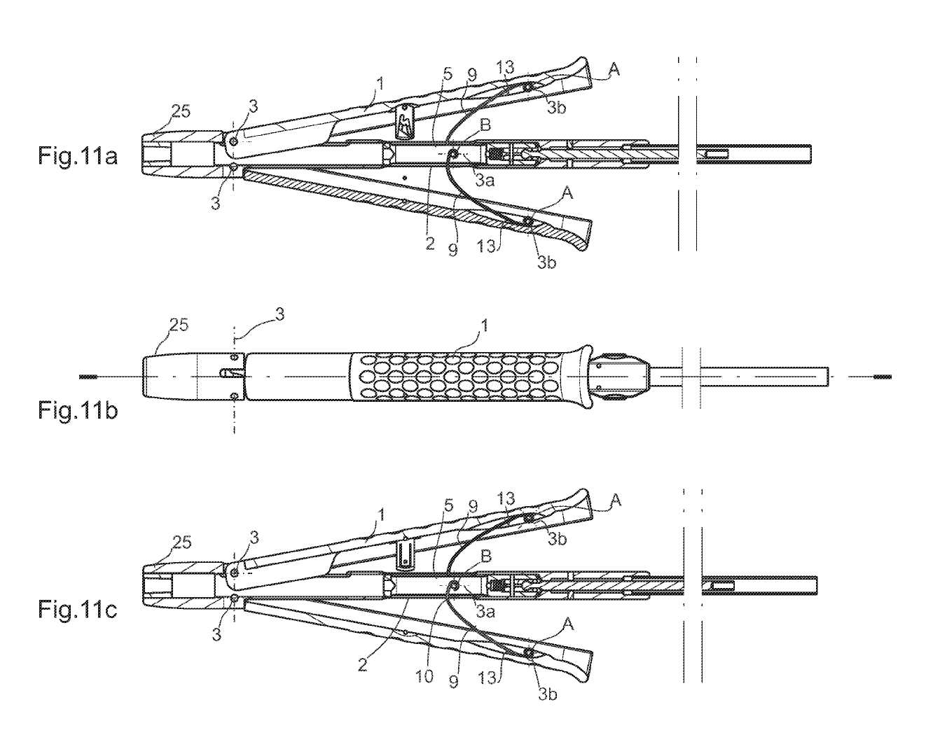

FIGS. 11a-11c illustrate a surgical handle comprising a leaf spring 9 according to the invention and an additional curvature 10 as overload protection in several views. At the proximal end of the surgical handle equally a Luer cone 25 may be arranged through which rinsing liquid can be guided. All further technical features correspond to the handle in accordance with FIG. 10.

1. A surgical handle of a shaft-type instrument for actuating a tool arranged distally on an instrument shaft, the surgical handle comprising:
a handle housing on which at least one lever/handle is mounted so as to be movable and pivoting relative to the handle housing, which lever/handle is coupled via a toggle to a sliding element that is axially movable in the handle housing in order for the sliding element to be moved as the lever/handle is actuated from the starting position thereof against the restoring force of a spring element,
the spring element being formed by the toggle, wherein the one spring end acts on the sliding element and the other spring element acts on the lever/handle,
the spring element being adjacent to a central portion between the two spring ends at the lever/handle and at a guide link which is integrally formed on the lever/handle so that upon actuation of the lever/handle, the spring element rolls off the lever/handle.

2. The surgical handle according to claim 1, wherein the toggle forming the spring element is a leaf spring.

3. The surgical handle according to claim 1, wherein the spring element includes an arc-shaped curvature in the relieved state.

4. The surgical handle according to claim 2, wherein the lever/handle includes means for force application onto the leaf spring so as to cause, upon actuation of the lever/handle, bending of the leaf spring in the central portion thereof, the force application means being a projection arranged on the lever/handle or being a guide link for the leaf spring that is integrally formed on the lever/handle.

5. The surgical handle of a surgical instrument according to claim 2, wherein the connection of the leaf spring relative to the sliding element is a hinge-like or fixed connection.

6. The surgical handle of a surgical instrument according to claim 2, wherein the connection of the leaf spring relative to the sliding element is configured to be permanent.

7. The surgical handle of a surgical instrument according to claim 2, wherein the leaf spring is made of spring steel.

8. The surgical handle of a surgical instrument according to claim 2, wherein the leaf spring is made of plastic material.

9. The surgical handle of a surgical instrument according to claim 2, wherein, in the area of the connection to the sliding element, the leaf spring has a further radius of curvature smaller than the residual radius of curvature of the arc shape which is directed so that an actuation of the at least one lever/handle beyond a predetermined closing position entails additional deformation or bulging of the leaf spring in the area of the modified radius of curvature.

10. The surgical handle of a surgical instrument according to claim 1, wherein the at least one lever/handle is made of plastic material.

11. The surgical handle of a surgical instrument according to claim 1, wherein the at least one lever/handle comprises several parts.

12. The surgical handle of a surgical instrument according to claim 1, wherein the at least one lever/handle has a shell shape.

13. The surgical handle of a surgical instrument according to claim 1, wherein the at least one lever/handle surrounds at least portions of the handle housing in the circumferential direction of the handle axis in a sleeve shape.

14. The surgical handle of a surgical instrument according to claim 1, wherein the spring element is adjacent to a central portion between the two spring ends at the lever/handle so that, upon actuation of the lever/handle, the spring element rolls off the lever/handle.

15. A surgical instrument having a tubular shaft comprising a tubular shaft-type tool, an instrument shaft at the distal end of which the tubular shaft-type tool is arranged, and a surgical handle according to claim 1 which is or can be arranged on the proximal end of the instrument shaft.
